(19) United States
(12) Patent Application Publication
(10) Pub. No.: US 2023/0265483 A1
(43) Pub. Date: Aug. 24, 2023

(54) METHODS FOR TESTING ANDEXANET POTENCY

(71) Applicant: Alexion Pharmaceuticals, Inc., Boston, MA (US)

(72) Inventors: Joel Freeberg, Albany, CA (US); Genmin Lu, Tyngsboro, MA (US); Pamela Conley, Palo Alto, CA (US)

(73) Assignee: Alexion Pharmaceuticals, Inc., Boston, MA (US)

(21) Appl. No.: 18/010,076

(22) PCT Filed: Jun. 10, 2021
(86) PCT No.: PCT/US21/36729

§ 371 Date: Dec. 13, 2022

Related U.S. Application Data
(60) Provisional application No. 63/039,795, filed on Jun. 16, 2020.

(51) Int. Cl. C12Q 1/56; G01N 33/52; G01N 33/86

(52) U.S. Cl. CPC C12Q 1/56; G01N 33/52; G01N 33/86

(57) ABSTRACT

The present disclosure relates kits and methods for measuring the potency of an andexanet sample in neutralizing a factor Xa inhibitor and restoring the activity of a wild-type factor Xa.

### CROSS-REFERENCE TO RELATED APPLICATIONS

This application claims the benefit of priority to U.S. Application No. 63/039,795, filed Jun. 16, 2020, which is incorporated by reference herein for all purposes.

### BACKGROUND

Andexanet alfa (coagulation factor Xa [recombinant] inactivated-zhzo) is a modified recombinant inactive form of factor Xa designed specifically to bind and sequester factor Xa inhibitors. Factor Xa inhibitors, e.g., apixaban, rivaroxaban, edoxaban, betrixaban, and enoxaparin, are commonly used anticoagulants. The use of such anticoagulants, however, is associated with acute major bleeding episodes. Andexanet alfa or andexanet is an effective antidote to prevent and stop such bleedings.

It is important that, prior to use, each batch of the andexanet is evaluated for its activity and potency.

### SUMMARY

The present disclosure provides compositions, kits and methods for measuring the potency (activity) of andexanet alfa in test samples. The potency can be quantitated based on the test sample's ability to restore the activity of human wild-type fXa in a mixture with a fXa inhibitor. The activity of the human fXa can be further calibrated with a fXa protein, such as from bovine or human, for which an international reference standard has been established.

One embodiment of the present disclosure provides a method for determining the activity of an andexanet sample, comprising admixing a test sample comprising andexanet with a mixture comprising a human factor Xa (HFXa) and a direct factor Xa (fXa) inhibitor, wherein the molar ratio of the FXa to the Xa inhibitor is from 0.2:1 to 0.3:1; adding a chromogenic fXa substrate to the mixture, wherein the chromogenic fXa substrate is able to release a chromophore upon reaction with the HFXa; detecting the amount of released chromophore; and calculating, from the amount of the released chromophore, the activity of the andexanet sample in releasing the HFXa from the direct fXa inhibitor.

Another embodiment provides a method for determining the activity of an andexanet sample, comprising: admixing a test sample comprising andexanet with a mixture comprising a bovine factor Xa (BFXa) and a direct factor Xa (fXa) inhibitor, wherein the molar ratio of the BFXa to the fXa inhibitor is from 0.15:1 to 0.25:1; adding a chromogenic fXa substrate to the mixture, wherein the chromogenic fXa substrate is able to release a chromophore upon reaction with the BFXa; detecting the amount of released chromophore; and calculating, from the amount of the released chromophore, the activity of the andexanet sample in releasing the BFXa from the direct fXa inhibitor.

### BRIEF DESCRIPTION OF THE DRAWINGS

Provided as embodiments of this disclosure are drawings which illustrate by exemplification only, and not limitation, wherein:

FIG. 1 compares the enzymatic activity of human fXa vs bovine fXa (Hyphen BFXa) for the cleavage of Spectrozyme-fXa substrate. Human fXa (0) or BFXa (□) was serially diluted in 1×TBS (pH7.4, 0.1% BSA) at 250, 125, 62.5, 31.25, 15.625, 7.8125, 3.90625, 0 ng/mL. The serially diluted fXa (100 μL) was mixed with 50 μL of Spectrozyme-fXa (2 mM). The reaction was quenched after 10 min incubation at room temperature by addition of 50 μL acetic acid. Bovine fXa has approximately 31% less activity than HFXa based on the slope (OD405 nm vs concentration).

FIG. 2A-C compare bovine fXa and human fXa by matching enzyme concentrations in the direct potency assay. 2A. BFXa (1×)/betrixaban (1×); 2B. BFXa (1×)/betrixaban (1.5×); 2C. BFXa (1×)/betrixaban (2×). The stock solutions for HFXa, BFXa and betrixaban were first prepared separately. FXa/betrixaban mixture was prepared by mixing different volumes of the stock solutions of FXa and betrixban, resulting in HFXa/betrixaban or BFXa/betrixaban. The FXa/betrixaban mixture (50 μL) was added to the reaction mixture as described in the direct potency assay. The HFXa (1×)/betrixaban (1×) mixture (O) was used as a control. The BFXa/betrixaban mixture (□) was tested at different concentrations of betrixaban, while keeping the same concentration of BFXa (1×). Panel a): BFXa (1×)/betrixaban (1×); Panel b: BFXa (1×)/betrixaban (1.5×); Panel c): BFXa (1×)/betrixaban (2×).

FIG. 3A-B show the optimization results of bovine FXa and betrixaban concentrations by matching the enzymatic activity. 3A. BFXa (1.64×)/betrixaban (2×); 3B. BFXa (1.45×)/betrixaban (2×). The stock solutions for HFXa, BFXa and betrixaban were first prepared separately. FXa/betrixaban mixture was prepared by mixing different volumes the stock solutions of FXa and betrixban, resulting in HFXa/betrixaban or BFXa/betrixaban. The FXa/betrixaban mixture (50 μL) was added to the reaction mixture as described in the direct potency assay. The HFXa (1×)/betrixaban (1×) mixture (O) was used as a control. The BFXa/betrixaban mixture (□) was tested at different concentrations of BFXa, while keeping the same concentration of betrixaban (2×). Panel a): BFXa (1.64×)/betrixaban (2×); Panel b: BFXa (1.45×)/betrixaban (2×).

### DETAILED DESCRIPTION

### Definitions

All numerical designations, e.g., pH, temperature, time, concentration, and molecular weight, including ranges, are approximations which are varied (+) or (−) by increments of 0.1. It is to be understood, although not always explicitly stated that all numerical designations are preceded by the term “about”. It also is to be understood, although not always explicitly stated, that the reagents described herein are merely exemplary and that equivalents of such are known in the art.

A “composition” is intended to mean a combination of active agent and another compound or composition, inert (for example, a detectable agent or label) or active.

“Factor Xa” or “fXa” or “fXa protein” is a serine protease in the blood coagulation pathway, which is produced from the inactive factor X (fX). The nucleotide sequence coding human factor X (“fX”) can be found in GenBank with accession number “NM 000504.” Upon catalytic cleavage of the first 52 residues of the heavy chain, fX is activated to fXa (SEQ ID NO. 1, Table 1). FXa contains a light chain and a heavy chain (as shown in Table 1). The first 45 amino acid residues (residues 1-45 of SEQ ID NO. 1) of the light chain is called the Gla domain because it contains 11 post-translationally modified γ-carboxyglutamic acid residues (Gla). It also contains a short (6 amino acid residues) aromatic stack sequence (residues 40-45 of SEQ ID NO. 1). Chymotrypsin digestion selectively removes the 1-44 residues resulting in Gla-domainless fXa. The serine protease catalytic domain of fXa is located on the C-terminal heavy chain. The heavy chain of fXa is highly homologous to other serine proteases such as thrombin, trypsin, and activated protein C.

“Native fXa” or “wild-type fXa” refers to the fXa naturally present in plasma or being isolated in its original, unmodified form, which possesses the biological activity of activating prothrombin therefore promoting formation of blood clot. The term includes naturally occurring polypeptides isolated from tissue samples as well as recombinantly produced fXa. “Active fXa” refers to fXa having the biological activity of activating prothrombin. “Active fXa” may be a native fXa or modified fXa that retains procoagulant activity.

As used herein, “fXa derivatives” refer to modified fXa proteins that have a modification or substantial deletion (e.g., deletion of at least 50%, 60%, 70%, 80%, or 90% of the amino acid residues 6-39 of the light chain) of the Gla-domain and a modification to the active site such that the fXa derivatives, as compared to the wild-type protein, have reduced ability in assembling into the prothrombinase complex and reduced or no catalytic activities. Still, similar to the wild-type protein, the fXa derivatives can bind and/or substantially neutralize fXa inhibitors. Examples of fXa derivatives are provided in U.S. Pat. Nos. 8,153,590 and 8,268,783, and biological equivalents thereof.

The term “active site” refers to the part of an enzyme or antibody where a chemical reaction occurs. A “modified active site” is an active site that has been modified structurally to provide the active site with increased or decreased chemical reactivity or specificity. Examples of active sites include, but are not limited to, the catalytic domain of human factor X comprising the 235-488 amino acid residues, and the catalytic domain of human factor Xa comprising the 195-448 amino acid residues. Examples of modified active site include, but are not limited to, the catalytic domain of human factor Xa comprising 195-448 amino acid residues in SEQ ID NOS. 2 with at least one amino acid substitution at position Arg306, Glu310, Arg347, Lys351, Lys414, or Arg424.

An example of a fXa derivative is a “Gla-domainless fXa” or “des-Gla fXa” that refers to fXa or a fXa derivative that does not have a Gla-domain and encompasses fXa derivatives bearing other modification(s) in addition to the removal of the Gla-domain. Examples of Gla-domainless fXa in this invention include, but are not limited to, fXa derivative lacking all or part of the 1-39 (or 6-39) amino acid residues of SEQ ID NO. 1.

Another example of a fXa derivative is a “Gla-deficient fXa” that refers to fXa or a fXa derivative with reduced number of free side chain γ-carboxyl groups in its Gla-domain. Like Gla-domainless fXa, Gla-deficient fXa can also bear other modifications. Gla-deficient fXa includes uncarboxylated, undercarboxylated and decarboxylated fXa. “Uncarboxylated fXa” or “decarboxylated fXa” refers to fXa derivatives that do not have the γ-carboxy groups of the γ-carboxyglutamic acid residues of the Gla domain, such as fXa having all of its Gla domain γ-carboxyglutamic acid replaced by different amino acids, or fXa having all of its side chain γ-carboxyl removed or masked by means such as amination, esterification, etc. For recombinantly expressed protein, uncarboxylated fXa is, sometimes, also called non-carboxylated fXa. “Undercarboxylated fXa” refers to fXa derivatives having reduced number of γ-carboxy groups in the Gla domain as compared with wild-type fXa, such as fXa having one or more but not all of its Gla domain γ-carboxyglutamic acids replaced by one or more different amino acids, or fXa having at least one but not all of its side chain γ-carboxyl removed or masked by means such as amination and esterification, etc.

In some embodiments, the fXa derivatives have a deletion of at least amino acid residues 6-39 of the light chain of the wild-type fXa (see light chain in SEQ ID NO:1). In some embodiments, the fXa derivatives also have a deletion of the EGF1 domain (amino acids 46-84) and/or the EGF2 domain (amino acids 85-128 of the light chain). In some embodiments, the light chain of the fXa derivative includes at least amino acids 129-139 of the wild-type light chain (or 95-105 of SEQ ID NO:3).

SEQ ID NO: 2 (Table 2), which is also referred to as “andexanet alfa” or simply “andexanet”, contains two mutations relative to the wild-type fXa”. The first mutation is the deletion of amino acid residues 6-39 in the Gla-domain of fX. The second mutation is mutation of active site residue 5379 to an Ala residue. This amino acid substitution corresponds to amino acid 296 of SEQ ID NOS: 1, respectively. Andexanet includes two chains, referred to as SEQ ID NO:3 (light chain) and 4 (heavy chain), respectively.

fXa derivatives also encompass those that contain mutations, post-translational modifications, and/or changes to the protein during the manufacturing process. For instance, in another aspect, the fXa derivatives, with or without the Ser379 modification, may contain modifications on the His (to Ala) and/or Asp (to Ala/Asn) residues in the catalytic triad, and a deleted or modified Gla domain. These modifications provide fXa derivatives with reduced enzymatic activity but not competing with fXa in assembling into the prothrombinase complex.

In some embodiments, the fXa derivative has a heavy chain that has one or two 0-linked glycosylation. In some embodiments, the heavy chain may have a C-terminal truncation of an amino acid residue, or up to 13, 14 or 15 amino acid residues. In some embodiments, the light chain may have an N-terminal truncation.

The present disclosure provides a variety of biological equivalents of the disclosed sequences of the fXa derivatives, or alternatively polypeptides having certain sequence identity to these fXa derivatives. In one aspect, such biological equivalents retain the structural characteristics of these fXa derivatives, that is, a modified active site or heavy chain and a deleted or modified Gla domain. In another aspect, such biological equivalents retain the functional features of these fXa derivatives, that is, not competing with fXa in assembling into the prothrombinase complex and having reduced catalytic activities. In another aspect, such biological equivalents can bind and/or substantially neutralize fXa inhibitors.

TABLE 1
Polypeptide sequence of activated human factor X, fXa (SEQ ID NO: 1)
Light Chain

| | |
|---|---|
| 1 | ANSFLEEMKK GHLERECMEE TCSYEEAREV FEDSDKTNEF WNKYKDGDQC ETSPCQNQGK |
| 61 | CKDGLGEYTC TCLEGFEGKN CELFTRKLCS LDNGDCDQFC HEEQNSVVCS CARGYTLADN |
| 121 | GKACIPTGPY PCGKQTLER |

Heavy Chain

| | |
|---|---|
| 181 | IVGGQE CKDGECPWQA LLINEENEGF CGGTILSEFY ILTAAHCLYQ |
| 241 | AKRFKVRVGD RNTEQEEGGE AVHEVEVVIK HNRFTKETYD FDIAVLRLKT PITFRMNVAP |
| 301 | ACLPERDWAE STLMTQKTGI VSGFGRTHEK GRQSTRLKML EVPYVDRNSC KLSSSFIITQ |
| 361 | NMFCAGYDTK QEDACQGDAG GPHVTRFKDT YFVTGIVSWG EGCARKGKYG IYTKVTAFLK |
| 421 | WIDRSMKTRG LPKAKSHAPE VITSSPLK |

TABLE 2
Polypeptide sequence of andexanet (SEQ ID NO: 2)
Light Chain (SEQ ID NO: 3)

| | |
|---|---|
| 1 | ANSFL F WNKYKDGDQC ETSPCQNQGK |
| 61 | CKDGLGEYTC TCLEGFEGKN CELFTRKLCS LDNGDCDQFC HEEQNSVVCS CARGYTLADN |
| 121 | GKACIPTGPY PCGKQTLER |

Heavy Chain (SEQ ID NO: 4)

| | |
|---|---|
| 181 | IVGGQE CKDGECPWQA LLINEENEGF CGGTILSEFY ILTAAHCLYQ |
| 241 | AKRFKVRVGD RNTEQEEGGE AVHEVEVVIK HNRFTKETYD FDIAVLRLKT PITFRMNVAP |
| 301 | ACLPERDWAE STLMTQKTGI VSGFGRTHEK GRQSTRLKML EVPYVDRNSC KLSSSFIITQ |
| 361 | NMFCAGYDTK QEDACQGDAG GPHVTRFKDT YFVTGIVSWG EGCARKGKYG IYTKVTAFLK |
| 421 | WIDRSMKTRG LPKAKSHAPE VITSSPLK |

The term “factor Xa inhibitors” or “inhibitors of factor Xa” refer to compounds that can inhibit, either directly or indirectly, the coagulation factor Xa's activity of catalyzing conversion of prothrombin to thrombin in vitro and/or in vivo.

“Direct factor Xa inhibitors” or “direct fXa inhibitors” bind to the fXa directly and inhibit its activity. Non-limiting examples include rivaroxaban (Xarelto), apixaban (Eliquis), betrixaban (Bevyxxa), darexaban (YM150), edoxaban (Lixiana), otamixaban, letaxaban (TAK-442), eribaxaban (PD0348292), and pharmaceutically acceptable salts thereof, and combinations thereof.

“Indirect factor Xa inhibitors” or “indirect fXa inhibitors” inhibit the fXa activity via one or more other factors. Non-limiting examples of indirect factor Xa inhibitors include enoxaparin, fondaparinux, idraparinux, biotinylated idraparinux, fragmin, tinzaparin, low molecular weight heparin (“LMWH”), and combinations thereof.

### II. Potency Assay

Anticoagulants serve a need in the marketplace in treatment or prevention of undesired thrombosis in patients with a tendency to form blood clots, such as, for example, those patients having clotting disorders, confined to periods of immobility or undergoing medical surgeries. One of the major limitations of anticoagulant therapy, however, is the bleeding risk associated with the treatments, and limitations on the ability to rapidly reverse the anticoagulant activity in case of overdosing or if an urgent surgical procedure is required.

Andexanet alfa (andexanet) is a recombinant modified human factor Xa decoy protein that can reverse the inhibition of factor Xa, and thus is an effective antidote to factor Xa-based anticoagulant treatments. Andexanet is manufactured as a recombinant protein produced from cultured cells. Prior to use, it is important to measure the potency of each production batch.

A potency measurement assay entails adding an andexanet sample to a fXa/inhibitor mixture in which the fXa has at least partially been inhibited by the inhibitor. Upon addition of the andexanet sample, the inhibitor is sequestered, releasing all or part of the bound fXa, which can be measured by virtue of its ability to cleave a fXa-specific chromogenic substrate, and release a chromophore.

Interestingly, it is discovered that a suitable fXa/inhibitor mixture requires a much higher number of inhibitor than fXa. For instance, when a human fXa was used together with a particular fXa inhibitor, betrixaban maleate, a suitable molar ratio was 0.25:1.

In certain situations, it is preferred that the measured andexanet potency is represented with a fXa that has been calibrated with an international standard. Unfortunately, the human fXa did not have a suitable international standard until recently. To this end, Example 2 explored the use of a bovine fXa.

Interestingly, the bovine fXa exhibited a considerably lower chromogenic activity. This presented a challenge. If the bovine fXa is matched by mass or concentration with the human fXa, the bovine fXa would have less activity. On the other hand, if the bovine fXa is matched activity, the bovine fXa would have molar excess over the human fXa and thus affect the aa/inhibitor ratio and the degree of inhibition in the assay. Yet another surprising finding is that betrixaban was less potent in inhibiting the bovine than the human fXa.

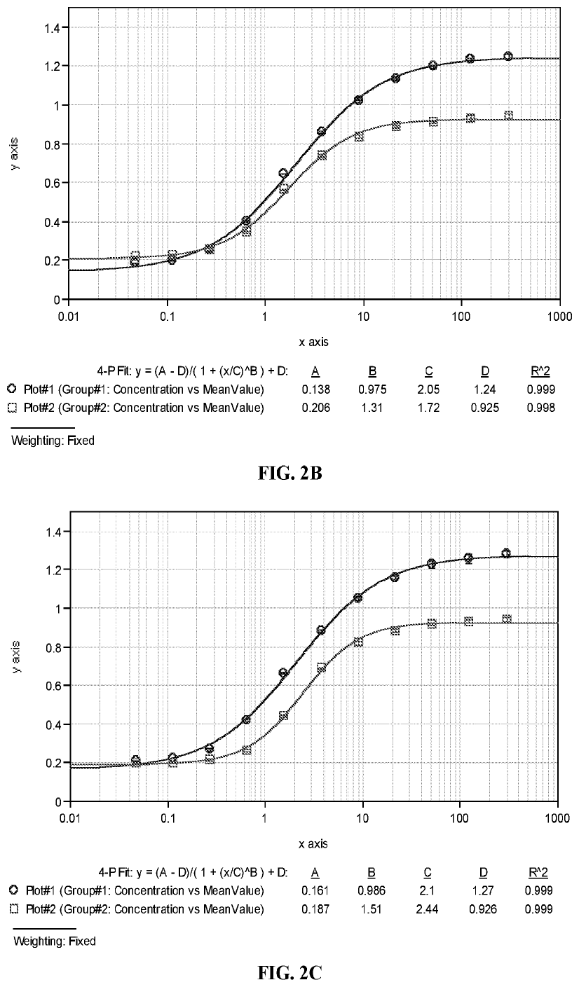

Based on these observations, the betrixaban concentration in the mixture was increased (1.5×, 2.0×) in order to reduce the background activity of BFXa in the absence of andexanet, while keeping the same concentration for BFXa (1×). As shown in FIG. 2(b-c). A 2-fold increase of betrixaban concentration reduced the BFXa background to the level similar to HFXa in the absence of andexanet. However, the maximum absorbance with BFXa (1×) was lower in the presence of the highest andexanet concentration than the control with HFXa (1×)/betrixaban (1×).

A surprising discovery of the present disclosure is that, despite the differences in chromogenic activities and the abilities of being inhibited by fXa inhibitor, comparable profiles between bovine and human fXa controls in terms of maximum and minimum absorbance, EC₅₀, and assay ranges could be achieved by adjusting the bovine fXa concentration in the mixture. In some embodiments, the bovine aa/inhibitor molar ratio may be around 0.18:1. In some embodiments, the bovine fXa concentration may be around 16.2 nM.

In accordance with one embodiment of the present disclosure, therefore, provided is a method for determining the activity of an andexanet sample. In some embodiments, the method entails admixing a test sample comprising andexanet with a mixture comprising a human factor Xa (HFXa) and a direct factor Xa (fXa) inhibitor. Then, a chromogenic fXa substrate is added to the mixture, wherein the chromogenic fXa substrate is able to release a chromophore upon reaction with the HFXa. Once the amount of released chromophore is detected, the activity of the andexanet sample can be calculated with respect to releasing the HFXa from the direct fXa inhibitor.

An “andexanet sample,” as used herein, refers to a sample that includes one or more andexanet molecules. An andexanet sample may be a particular volume or mass of a sample, taken from a larger volume or mass that has been prepared, such as through pharmaceutical manufacturing, or prior to clinical use.

In some embodiments, the molar ratio of the fXa to the fXa inhibitor in the mixture is from 0.2:1 to 0.3:1. In some embodiments, the molar ratio of the fXa to the fXa inhibitor in the mixture is from 0.1:1 to 0.5:1, 0.12:1 to 0.5:1, 0.14:1 to 0.5:1, 0.15:1 to 0.5:1, 0.16:1 to 0.5:1, 0.18:1 to 0.5:1, 0.2:1 to 0.5:1, 0.22:1 to 0.5:1, 0.24:1 to 0.5:1, 0.25:1 to 0.5:1, 0.26:1 to 0.5:1, 0.28:1 to 0.5:1, 0.3:1 to 0.5:1, 0.1:1 to 0.48:1, 0.12:1 to 0.48:1, 0.14:1 to 0.48:1, 0.15:1 to 0.48:1, 0.16:1 to 0.48:1, 0.18:1 to 0.48:1, 0.2:1 to 0.48:1, 0.22:1 to 0.48:1, 0.24:1 to 0.48:1, 0.25:1 to 0.48:1, 0.26:1 to 0.48:1, 0.28:1 to 0.48:1, 0.3:1 to 0.48:1, 0.1:1 to 0.46:1, 0.12:1 to 0.46:1, 0.14:1 to 0.46:1, 0.15:1 to 0.46:1, 0.16:1 to 0.46:1, 0.18:1 to 0.46:1, 0.2:1 to 0.46:1, 0.22:1 to 0.46:1, 0.24:1 to 0.46:1, 0.25:1 to 0.46:1, 0.26:1 to 0.46:1, 0.28:1 to 0.46:1, 0.3:1 to 0.46:1, 0.1:1 to 0.45:1, 0.12:1 to 0.45:1, 0.14:1 to 0.45:1, 0.15:1 to 0.45:1, 0.16:1 to 0.45:1, 0.18:1 to 0.45:1, 0.2:1 to 0.45:1, 0.22:1 to 0.45:1, 0.24:1 to 0.45:1, 0.25:1 to 0.45:1, 0.26:1 to 0.45:1, 0.28:1 to 0.45:1, or 0.3:1 to 0.45:1.

In some embodiments, the molar ratio of the fXa to the fXa inhibitor in the mixture is from 0.1:1 to 0.44:1, 0.12:1 to 0.44:1, 0.14:1 to 0.44:1, 0.15:1 to 0.44:1, 0.16:1 to 0.44:1, 0.18:1 to 0.44:1, 0.2:1 to 0.44:1, 0.22:1 to 0.44:1, 0.24:1 to 0.44:1, 0.25:1 to 0.44:1, 0.26:1 to 0.44:1, 0.28:1 to 0.44:1, 0.3:1 to 0.44:1, 0.1:1 to 0.42:1, 0.12:1 to 0.42:1, 0.14:1 to 0.42:1, 0.15:1 to 0.42:1, 0.16:1 to 0.42:1, 0.18:1 to 0.42:1, 0.2:1 to 0.42:1, 0.22:1 to 0.42:1, 0.24:1 to 0.42:1, 0.25:1 to 0.42:1, 0.26:1 to 0.42:1, 0.28:1 to 0.42:1, 0.3:1 to 0.42:1, 0.1:1 to 0.4:1, 0.12:1 to 0.4:1, 0.14:1 to 0.4:1, 0.15:1 to 0.4:1, 0.16:1 to 0.4:1, 0.18:1 to 0.4:1, 0.2:1 to 0.4:1, 0.22:1 to 0.4:1, 0.24:1 to 0.4:1, 0.25:1 to 0.4:1, 0.26:1 to 0.4:1, 0.28:1 to 0.4:1, 0.3:1 to 0.4:1, 0.1:1 to 0.38:1, 0.12:1 to 0.38:1, 0.14:1 to 0.38:1, 0.15:1 to 0.38:1, 0.16:1 to 0.38:1, 0.18:1 to 0.38:1, 0.2:1 to 0.38:1, 0.22:1 to 0.38:1, 0.24:1 to 0.38:1, 0.25:1 to 0.38:1, 0.26:1 to 0.38:1, 0.28:1 to 0.38:1, or 0.3:1 to 0.38:1.

In some embodiments, the molar ratio of the fXa to the fXa inhibitor in the mixture is from 0.1:1 to 0.36:1, 0.12:1 to 0.36:1, 0.14:1 to 0.36:1, 0.15:1 to 0.36:1, 0.16:1 to 0.36:1, 0.18:1 to 0.36:1, 0.2:1 to 0.36:1, 0.22:1 to 0.36:1, 0.24:1 to 0.36:1, 0.25:1 to 0.36:1, 0.26:1 to 0.36:1, 0.28:1 to 0.36:1, 0.3:1 to 0.36:1, 0.1:1 to 0.35:1, 0.12:1 to 0.35:1, 0.14:1 to 0.35:1, 0.15:1 to 0.35:1, 0.16:1 to 0.35:1, 0.18:1 to 0.35:1, 0.2:1 to 0.35:1, 0.22:1 to 0.35:1, 0.24:1 to 0.35:1, 0.25:1 to 0.35:1, 0.26:1 to 0.35:1, 0.28:1 to 0.35:1, 0.3:1 to 0.35:1, 0.1:1 to 0.34:1, 0.12:1 to 0.34:1, 0.14:1 to 0.34:1, 0.15:1 to 0.34:1, 0.16:1 to 0.34:1, 0.18:1 to 0.34:1, 0.2:1 to 0.34:1, 0.22:1 to 0.34:1, 0.24:1 to 0.34:1, 0.25:1 to 0.34:1, 0.26:1 to 0.34:1, 0.28:1 to 0.34:1, 0.3:1 to 0.34:1, 0.1:1 to 0.32:1, 0.12:1 to 0.32:1, 0.14:1 to 0.32:1, 0.15:1 to 0.32:1, 0.16:1 to 0.32:1, 0.18:1 to 0.32:1, 0.2:1 to 0.32:1, 0.22:1 to 0.32:1, 0.24:1 to 0.32:1, 0.25:1 to 0.32:1, 0.26:1 to 0.32:1, 0.28:1 to 0.32:1, or 0.3:1 to 0.32:1.

In some embodiments, the molar ratio of the fXa to the fXa inhibitor in the mixture is from 0.1:1 to 0.3:1, 0.12:1 to 0.3:1, 0.14:1 to 0.3:1, 0.15:1 to 0.3:1, 0.16:1 to 0.3:1, 0.18:1 to 0.3:1, 0.2:1 to 0.3:1, 0.22:1 to 0.3:1, 0.24:1 to 0.3:1, 0.25:1 to 0.3:1, 0.26:1 to 0.3:1, 0.28:1 to 0.3:1, 0.1:1 to 0.28:1, 0.12:1 to 0.28:1, 0.14:1 to 0.28:1, 0.15:1 to 0.28:1, 0.16:1 to 0.28:1, 0.18:1 to 0.28:1, 0.2:1 to 0.28:1, 0.22:1 to 0.28:1, 0.24:1 to 0.28:1, 0.25:1 to 0.28:1, 0.26:1 to 0.28:1, 0.1:1 to 0.26:1, 0.12:1 to 0.26:1, 0.14:1 to 0.26:1, 0.15:1 to 0.26:1, 0.16:1 to 0.26:1, 0.18:1 to 0.26:1, 0.2:1 to 0.26:1, 0.22:1 to 0.26:1, 0.24:1 to 0.26:1, 0.25:1 to 0.26:1, 0.1:1 to 0.25:1, 0.12:1 to 0.25:1, 0.14:1 to 0.25:1, 0.15:1 to 0.25:1, 0.16:1 to 0.25:1, 0.18:1 to 0.25:1, 0.2:1 to 0.25:1, 0.22:1 to 0.25:1, 0.24:1 to 0.25:1, 0.1:1 to 0.24:1, 0.12:1 to 0.24:1, 0.14:1 to 0.24:1, 0.15:1 to 0.24:1, 0.16:1 to 0.24:1, 0.18:1 to 0.24:1, 0.2:1 to 0.24:1, 0.22:1 to 0.24:1, 0.1:1 to 0.22:1, 0.12:1 to 0.22:1, 0.14:1 to 0.22:1, 0.15:1 to 0.22:1, 0.16:1 to 0.22:1, 0.18:1 to 0.22:1, 0.2:1 to 0.22:1, 0.1:1 to 0.2:1, 0.12:1 to 0.2:1, 0.14:1 to 0.2:1, 0.15:1 to 0.2:1, 0.16:1 to 0.2:1, or 0.18:1 to 0.2:1.

In some embodiments, the molar ratio of the fXa to the fXa inhibitor in the mixture is from 0.21 to 0.29, 0.22 to 0.28, 0.23 to 0.27, 0.24 to 0.26, 0.245 to 0.255, 0.248 to 0.252, or about 0.25, without limitation.

In some embodiments, the concentration of the HFXa, after the test sample is added, about 8 to 14 nM. In some embodiments, the concentration is from 5 to 20 nM, 6 to 20 nM, 7 to 20 nM, 8 to 20 nM, 9 to 20 nM, 10 to 20 nM, 11 to 20 nM, 12 to 20 nM, 5 to 19 nM, 6 to 19 nM, 7 to 19 nM, 8 to 19 nM, 9 to 19 nM, 10 to 19 nM, 11 to 19 nM, 12 to 19 nM, 5 to 18 nM, 6 to 18 nM, 7 to 18 nM, 8 to 18 nM, 9 to 18 nM, 10 to 18 nM, 11 to 18 nM, 12 to 18 nM, 5 to 17 nM, 6 to 17 nM, 7 to 17 nM, 8 to 17 nM, 9 to 17 nM, 10 to 17 nM, 11 to 17 nM, 12 to 17 nM, 5 to 16 nM, 6 to 16 nM, 7 to 16 nM, 8 to 16 nM, 9 to 16 nM, 10 to 16 nM, 11 to 16 nM, 12 to 16 nM, 5 to 15 nM, 6 to 15 nM, 7 to 15 nM, 8 to 15 nM, 9 to 15 nM, 10 to 15 nM, 11 to 15 nM, 12 to 15 nM, 5 to 14 nM, 6 to 14 nM, 7 to 14 nM, 8 to 14 nM, 9 to 14 nM, 10 to 14 nM, 11 to 14 nM, 12 to 14 nM, 5 to 13 nM, 6 to 13 nM, 7 to 13 nM, 8 to 13 nM, 9 to 13 nM, 10 to 13 nM, 11 to 13 nM, 12 to 13 nM, 5 to 12 nM, 6 to 12 nM, 7 to 12 nM, 8 to 12 nM, 9 to 12 nM, 10 to 12 nM, 11 to 12 nM, 5 to 11 nM, 6 to 11 nM, 7 to 11 nM, 8 to 11 nM, 9 to 11 nM, 10 to 11 nM, 5 to 10 nM, 6 to 10 nM, 7 to 10 nM, 8 to 10 nM, or 9 to 10 nM.

It is contemplated that any direct fXa inhibitor can be used for the assay. In some embodiments, the direct fXa inhibitor is selected from the group consisting of betrixaban, apixaban, rivaroxaban, edoxaban, otamixaban, letaxaban and eribaxaban. In some embodiments, the direct fXa inhibitor is betrixaban. In some embodiments, the direct fXa inhibitor is apixaban. In some embodiments, the direct fXa inhibitor is rivaroxaban.

It can be helpful, in some embodiments, that the method further entails validating the activity of the andexanet sample with a standard curve generated with a reference andexanet sample.

Another embodiment of the present disclosure provides a method for determining the activity of an andexanet sample, using a fXa protein that has an established international standard. In some embodiments, such a fXa is bovine fXa (BFXa). In some embodiments, such a fXa is human fXa (HFXa). It is known that the human fXa (HFXa) has a molecular weight of about 46 kDa, and the bovine fXa (BFXa) has a molecular weight of about 45.3 kDa.

Accordingly, in some embodiments, the method entails admixing a test sample comprising andexanet with a mixture comprising a bovine factor Xa (BFXa) and a direct factor Xa (fXa) inhibitor. Subsequently, a chromogenic fXa substrate is added to the mixture, wherein the chromogenic fXa substrate is able to release a chromophore upon reaction with the BFXa. The amount of released chromophore is detected which can be used to calculate the activity of the andexanet sample in releasing the BFXa from the direct fXa inhibitor.

In some embodiments, the molar ratio of the BFXa to the fXa inhibitor is from about 0.15:1 to 0.25:1. In some embodiments, the molar ratio of the BFXa to the fXa inhibitor is from about 0.1:1 to 0.5:1, 0.12:1 to 0.5:1, 0.14:1 to 0.5:1, 0.15:1 to 0.5:1, 0.16:1 to 0.5:1, 0.18:1 to 0.5:1, 0.2:1 to 0.5:1, 0.22:1 to 0.5:1, 0.24:1 to 0.5:1, 0.25:1 to 0.5:1, 0.1:1 to 0.48:1, 0.12:1 to 0.48:1, 0.14:1 to 0.48:1, 0.15:1 to 0.48:1, 0.16:1 to 0.48:1, 0.18:1 to 0.48:1, 0.2:1 to 0.48:1, 0.22:1 to 0.48:1, 0.24:1 to 0.48:1, 0.25:1 to 0.48:1, 0.1:1 to 0.46:1, 0.12:1 to 0.46:1, 0.14:1 to 0.46:1, 0.15:1 to 0.46:1, 0.16:1 to 0.46:1, 0.18:1 to 0.46:1, 0.2:1 to 0.46:1, 0.22:1 to 0.46:1, 0.24:1 to 0.46:1, 0.25:1 to 0.46:1, 0.1:1 to 0.45:1, 0.12:1 to 0.45:1, 0.14:1 to 0.45:1, 0.15:1 to 0.45:1, 0.16:1 to 0.45:1, 0.18:1 to 0.45:1, 0.2:1 to 0.45:1, 0.22:1 to 0.45:1, 0.24:1 to 0.45:1, 0.25:1 to 0.45:1, 0.1:1 to 0.44:1, 0.12:1 to 0.44:1, 0.14:1 to 0.44:1, 0.15:1 to 0.44:1, 0.16:1 to 0.44:1, 0.18:1 to 0.44:1, 0.2:1 to 0.44:1, 0.22:1 to 0.44:1, 0.24:1 to 0.44:1, or 0.25:1 to 0.44:1.

In some embodiments, the molar ratio of the BFXa to the fXa inhibitor is from about 0.1:1 to 0.42:1, 0.12:1 to 0.42:1, 0.14:1 to 0.42:1, 0.15:1 to 0.42:1, 0.16:1 to 0.42:1, 0.18:1 to 0.42:1, 0.2:1 to 0.42:1, 0.22:1 to 0.42:1, 0.24:1 to 0.42:1, 0.25:1 to 0.42:1, 0.1:1 to 0.4:1, 0.12:1 to 0.4:1, 0.14:1 to 0.4:1, 0.15:1 to 0.4:1, 0.16:1 to 0.4:1, 0.18:1 to 0.4:1, 0.2:1 to 0.4:1, 0.22:1 to 0.4:1, 0.24:1 to 0.4:1, 0.25:1 to 0.4:1, 0.1:1 to 0.38:1, 0.12:1 to 0.38:1, 0.14:1 to 0.38:1, 0.15:1 to 0.38:1, 0.16:1 to 0.38:1, 0.18:1 to 0.38:1, 0.2:1 to 0.38:1, 0.22:1 to 0.38:1, 0.24:1 to 0.38:1, 0.25:1 to 0.38:1, 0.1:1 to 0.36:1, 0.12:1 to 0.36:1, 0.14:1 to 0.36:1, 0.15:1 to 0.36:1, 0.16:1 to 0.36:1, 0.18:1 to 0.36:1, 0.2:1 to 0.36:1, 0.22:1 to 0.36:1, 0.24:1 to 0.36:1, 0.25:1 to 0.36:1, 0.1:1 to 0.35:1, 0.12:1 to 0.35:1, 0.14:1 to 0.35:1, 0.15:1 to 0.35:1, 0.16:1 to 0.35:1, 0.18:1 to 0.35:1, 0.2:1 to 0.35:1, 0.22:1 to 0.35:1, 0.24:1 to 0.35:1, 0.25:1 to 0.35:1, 0.1:1 to 0.34:1, 0.12:1 to 0.34:1, 0.14:1 to 0.34:1, 0.15:1 to 0.34:1, 0.16:1 to 0.34:1, 0.18:1 to 0.34:1, 0.2:1 to 0.34:1, 0.22:1 to 0.34:1, 0.24:1 to 0.34:1, 0.25:1 to 0.34:1, 0.1:1 to 0.32:1, 0.12:1 to 0.32:1, 0.14:1 to 0.32:1, 0.15:1 to 0.32:1, 0.16:1 to 0.32:1, 0.18:1 to 0.32:1, 0.2:1 to 0.32:1, 0.22:1 to 0.32:1, 0.24:1 to 0.32:1, or 0.25:1 to 0.32:1, 0.1:1 to 0.3:1, 0.12:1 to 0.3:1, 0.14:1 to 0.3:1, 0.15:1 to 0.3:1, 0.16:1 to 0.3:1, 0.18:1 to 0.3:1, 0.2:1 to 0.3:1, 0.22:1 to 0.3:1, 0.24:1 to 0.3:1, 0.25:1 to 0.3:1, 0.1:1 to 0.28:1, 0.12:1 to 0.28:1, 0.14:1 to 0.28:1, 0.15:1 to 0.28:1, 0.16:1 to 0.28:1, 0.18:1 to 0.28:1, 0.2:1 to 0.28:1, 0.22:1 to 0.28:1, 0.24:1 to 0.28:1, 0.25:1 to 0.28:1, 0.1:1 to 0.26:1, 0.12:1 to 0.26:1, 0.14:1 to 0.26:1, 0.15:1 to 0.26:1, 0.16:1 to 0.26:1, 0.18:1 to 0.26:1, 0.2:1 to 0.26:1, 0.22:1 to 0.26:1, 0.24:1 to 0.26:1, 0.25:1 to 0.26:1, 0.1:1 to 0.25:1, 0.12:1 to 0.25:1, 0.14:1 to 0.25:1, 0.15:1 to 0.25:1, 0.16:1 to 0.25:1, 0.18:1 to 0.25:1, 0.2:1 to 0.25:1, 0.22:1 to 0.25:1, 0.24:1 to 0.25:1, 0.1:1 to 0.24:1, 0.12:1 to 0.24:1, 0.14:1 to 0.24:1, 0.15:1 to 0.24:1, 0.16:1 to 0.24:1, 0.18:1 to 0.24:1, 0.2:1 to 0.24:1, 0.22:1 to 0.24:1, 0.1:1 to 0.22:1, 0.12:1 to 0.22:1, 0.14:1 to 0.22:1, 0.15:1 to 0.22:1, 0.16:1 to 0.22:1, 0.18:1 to 0.22:1, 0.2:1 to 0.22:1, 0.1:1 to 0.2:1, 0.12:1 to 0.2:1, 0.14:1 to 0.2:1, 0.15:1 to 0.2:1, 0.16:1 to 0.2:1, 0.18:1 to 0.2:1,

In some embodiments, the molar ratio of the BFXa to the fXa inhibitor is from about 0.15:1 to 0.25:1, 0.16:1 to 0.23:1, 0.16:1 to 0.23:1, 0.16:1 to 0.20:1, 0.16:1 to 0.19:1, 017:1 to 0.19:1, or about 0.18:1.

In some embodiments, the concentration of the BFXa, after the test sample is added, is about 10 to 20 nM. In some embodiments, the concentration of the BFXa, after the test sample is added, is about 10 to 20 nM, 11 to 20 nM, 12 to 20 nM, 13 to 20 nM, 14 to 20 nM, 15 to 20 nM, 16 to 20 nM, 17 to 20 nM, 10 to 19 nM, 11 to 19 nM, 12 to 19 nM, 13 to 19 nM, 14 to 19 nM, 15 to 19 nM, 16 to 19 nM, 17 to 19 nM, 10 to 18 nM, 11 to 18 nM, 12 to 18 nM, 13 to 18 nM, 14 to 18 nM, 15 to 18 nM, 16 to 18 nM, 17 to 18 nM, 10 to 17 nM, 11 to 17 nM, 12 to 17 nM, 13 to 17 nM, 14 to 17 nM, 15 to 17 nM, 16 to 17 nM, 10 to 16 nM, 11 to 16 nM, 12 to 16 nM, 13 to 16 nM, 14 to 16 nM, 15 to 16 nM, 10 to 15 nM, 11 to 15 nM, 12 to 15 nM, 13 to 15 nM, or 14 to 15 nM.

In some embodiments, the concentration of the BFXa, after the test sample is added, is about 14 to 18 nM, 15 to 17 nM, or 15.5 to 16.5 nM.

It is contemplated that any direct fXa inhibitor can be used for the assay. In some embodiments, the direct fXa inhibitor is selected from the group consisting of betrixaban, apixaban, rivaroxaban, edoxaban, otamixaban, letaxaban and eribaxaban. In some embodiments, the direct fXa inhibitor is betrixaban. In some embodiments, the direct fXa inhibitor is apixaban. In some embodiments, the direct fXa inhibitor is rivaroxaban.

It can be helpful, in some embodiments, that the method further entails validating the activity of the andexanet sample with a standard curve generated with a reference andexanet sample.

Another embodiment of the present disclosure provides a method for determining the activity of an andexanet sample, using a fXa protein that has an established international standard. In some embodiments, such a fXa is bovine fXa (BFXa). It is known that the human fXa (HFXa) has a molecular weight of about 46 kDa, and the bovine fXa (BFXa) has a molecular weight of about 45.3 kDa.

Accordingly, in some embodiments, the method entails admixing a test sample comprising andexanet with a mixture comprising a bovine factor Xa (BFXa) and a direct factor Xa (fXa) inhibitor. Subsequently, a chromogenic fXa substrate is added to the mixture, wherein the chromogenic fXa substrate is able to release a chromophore upon reaction with the BFXa. The amount of released chromophore is detected which can be used to calculate the activity of the andexanet sample in releasing the BFXa from the direct fXa inhibitor.

In some embodiments, the chromogenic fXa substrate is Spectrozyme-Xa.

In some embodiments, a method is provided for calibrating the activity of a human fXa (HFXa) protein by testing a reference andexanet with both the HFXa and a bovine fXa (BFXa). The testing results can then be used to represent the HFXa activity with the BFXa activity.

### EXAMPLES

The disclosure is further understood by reference to the following examples, which are intended to be purely exemplary of the disclosure. The present disclosure is not limited in scope by the exemplified embodiments, which are intended as illustrations of single aspects of the disclosure only. Any methods that are functionally equivalent are within the scope of the disclosure. Various modifications of the disclosure in addition to those described herein will become apparent to those skilled in the art from the foregoing description and accompanying figures. Such modifications fall within the scope of the appended claims.

### Example 1. Testing of Andexanet Sample Potency

This example describes an assay to determine the potency of andexanet alfa samples in neutralizing factor Xa inhibitors.

This assay measures the identity and potency of andexanet alfa based on its ability to bind to a direct fXa inhibitor, betrixaban maleate, and reverse the inhibition of human fXa in an assay mixture composed of human fXa and betrixaban. The restored human fXa activity is measured with a fXa-specific chromogenic substrate, which releases a chromophore upon cleavage by fXa. Test sample potency is determined by comparing the test sample response to the response obtained for a reference standard in a software program using the equivalence testing with the difference of the slopes of the 4-parameter curve fit. The potency is calculated by the ratio of the EC₅₀ of the sample vs the reference.

The process is as follows:
1. Remove an Aliquot of Human fXa and fXa Inhibitor (e.g., Betrixaban, Stock Solution 226 μg/mL) and thaw at room temperature
2. Preparation of fXa Reference Standard (RS), Assay Control and Test Samples (TS)
   a) Prepare duplicate dilutions of reference standard and each test sample at 0.6 mg/mL in assay buffer. Use actual measured concentration at the time of release for reference standard and use label claim (target) concentration of all test samples, unless otherwise specified. See examples below for final bulk at 10.0 mg/mL
   Example dilution
      Amount of TS needed=(0.6 mg/mL×1000 μL)/10.0 mg/mL=60.0 μL
      Amount of assay buffer needed=1000 μL−60 μL=940 μL
   b) Prepare serial dilutions of the reference standard (RS), assay control (CTRL), and test sample (TS) in the dilution microplate by performing 2.4-fold serial dilutions. See below for an example dilution scheme:

Example Serial Dilutions:

| | | Volume of | Target | Final |
|---|---|---|---|---|
| Dilution | | Assay Buffer | Conc. | Conc. |
| No. | Volume of Sample (μL) | (μL) | (μg/mL) | (μg/mL) |
| 1 | 300 of dilution at | 0 | 600 | 300 |
| | 0.6 mg/mL | | | |
| 2 | 125 of Dilution 1 | 175 | 250 | 125 |
| 3 | 125 of Dilution 2 | 175 | 104 | 52.1 |
| 4 | 125 of Dilution 3 | 175 | 43.4 | 21.7 |
| 5 | 125 of Dilution 4 | 175 | 18.1 | 9.04 |
| 6 | 125 of Dilution 5 | 175 | 7.54 | 3.77 |
| 7 | 125 of Dilution 6 | 175 | 3.14 | 1.57 |
| 8 | 125 of Dilution 7 | 175 | 1.31 | 0.654 |
| 19 | 125 of Dilution 8 | 175 | 0.S45 | 0.273 |
| 10 | 125 of Dilution 9 | 175 | 0.227 | 0.114 |
| 11 | 125 of Dilution 10 | 175 | 0.095 | 0.047 |

3. Preparation of fXa and Betrixaban (fXa Inhibitor)
   a) Dilute the fXa to 50 μg/mL then further dilute to 1.0 μg/mL in assay buffer.
   b) Dilute fXa inhibitor (226 μg/mL betrixaban stock) to 40 ng/mL in assay buffer as described in the table below.
   c) Add equal volumes of diluted fXa and fXa inhibitor (betrixaban) into an appropriate container by combining 6 mL of FXa at 1.0 μg/mL and 6 mL of fXa inhibitor at 40 ng/mL. Mix well.
4. Preparation of fXa and fXa Inhibitor Controls
   a) Prepare the fXa control by mixing 100 μL of diluted fXa with 300 μL of assay buffer.
   b) Prepare fXa inhibitor (betrixaban) control by mixing 200 μL of the fXa inhibitor mixture and 200 μL assay buffer.
5. Transfer 50 μL/well of reference standard (RS), assay control (CTRL) and test samples (TS) to the assay plate.
6. Add 50 μL of the human aa/inhibitor mixture (prepared in Step 3(c)) to all RS, CTRL, TS-1, TS-2 wells.
7. Transfer 100 μL/well of fXa control and aa/inhibitor control to the assay plate. Add 100 μL of assay buffer to each of the blank wells.
8. Shake the plate for approximately 1 minute on an orbital plate shaker set at −300 rpm to mix.
9. Cover the plate with an adhesive plate sealer and incubate for 60±5 minutes in the dark.
10. While the samples are incubating, thaw and prepare the Spectrozyme fXa substrate at a concentration of 2.0 mM. Add 4.5 mL of assay buffer into the thawed 3.0 mL single-use aliquot of Spectrozyme fXa substrate and mix well. Prepare this reagent immediately prior to use. This volume is sufficient for 1 assay plate.
11. Add 50 μL of the prepared Spectrozyme FXa substrate to all wells, transfer the plate immediately to a plate shaker set at −300 rpm for approximately 1 minute.
12. Incubate plate for 10±1 minutes in the dark, then stop the reaction with the addition of 50 μL of stop solution.
13. Read the microplate at 405 nm as the test wavelength and 490 nm as the reference wavelength.
14. Calculate potency of the test andexanet sample

### Example 2. Calibration of Potency Unit

The potency assay examined in Example 1 was developed based on andexanet's ability to bind to a direct fXa inhibitor (betrixaban) and reverse its inhibition of human fXa (HFXa) activity in an assay mixture composed of andexanet, HFXa and betrixaban. The restored HFXa activity is measured with a fXa-specific chromogenic substrate, which releases a chromophore upon cleavage by fXa. The reversal activity of the test sample is compared to an andexanet reference standard to obtain the specific activity (expressed either as % or mg/mg). The reference standard used in Example 1 was a HFXa, whose potency was not traceable to an independent international reference standard because none HFXa international reference standard (e.g., WHO) was established until more recenity.

This example describes the development of a direct potency unit assay using bovine BFXa (Hyphen-BioMed) in replacement of HFXa. BFXa activity has been calibrated against the international reference standard National Institute for Biological Standard and Control (NIBSC) (UK) [NIBSC Code: 75/595].

### Materials and Methods

Human FXa (HFXa) was purchased from Haematologic Technologies, Inc. (Essex Junction, VT) (Cat #HCXA-0060). A 2×working stock at 1.0 μg/mL was freshly made in Tris-buffered saline (1×TBS, pH 7.4, 0.1% BSA).

BFXa reference standard was purchased from Hyphen BioMed (France) (Cat #: BE1010/BE101K). Each vial of Hyphen BFXa contained 50 μg purified bovine FXa, or 111 Units/vial (i.e. 2.2 Units/μg or 0.45 μg/Unit) calibrated against NIBSC BFXa (75/595), the non-WHO international reference standard. Each vial was reconstituted with 1 mL H₂O as recommended, resulting in 50 μg/mL or 111 Units/mL. A working stock was made at 0.9 μg/mL (2.0 U/mL) in 1×TBS (pH 7.4, 0.1% BSA).

FXa inhibitor (betrixaban) was provided as 1 mM (452 μg/mL, mw: 425 free base) DMSO stock solution. A 2×working stock at 40 ng/mL was made in 1×TBS (pH 7.4, 0.1% BSA) to be mixed with HFXa. Alternative working stocks at 400, 300, 200, 40 ng/mL were made to be mixed with BFXa when varying betrixaban concentrations in the mixture.

FXa substrate Spectrozyme-Xa substrate was purchased from Sekisui Diagnostics (Lexington, Mass.) (Cat #222L). One vial of Spectrozyme-FXa (50 μmoles/vial) was reconstituted with 10 mL H2O, resulting in 5 mM stock solution. A 2 mM working stock was prepared in 1×TBS (pH 7.4, 0.1% BSA).

Andexanet alfa (50 mg/vial) was reconstituted with 4.7 mL H₂O as recommended, resulting in 9.7 mg/mL protein concentration by absorbance (A₂₈₀). Small aliquots were stored at −80° C. A working stock was made at 0.6 mg/mL in 1×TBS (pH 7.4, 0.1% BSA) and used to prepare the standards between 0-600 μg/mL.

### Results

The current direct potency assay used for release and stability testing of andexanet samples is an endpoint method by mixing equal volumes (50 μL) of each reagent. Human FXa and betrixaban (inhibitor) are first prepared as 2× stocks at 1.0 μg/mL (HFXa) and 40 ng/mL (betrixaban), and combined with equal volume (mixed 1:1), resulting in 0.5 μg/mL HFXa, 20 ng/mL betrixaban, respectively, in the mixture. The HFXa/inhibitor mixture (50 μL) is then added to the reaction mixture.

The specific activity Hyphen BFXa was calibrated against the NIBSC BFXa (75/595) reference standard by the manufacturer. The reconstituted stock concentration (50 μg/mL, 111 Units/mL, 0.45 μg/Unit) was used as the working reference standard in the direct potency units assay development.

### Comparison of the Chromogenic Activity Between HFXa and Hyphen BFXa

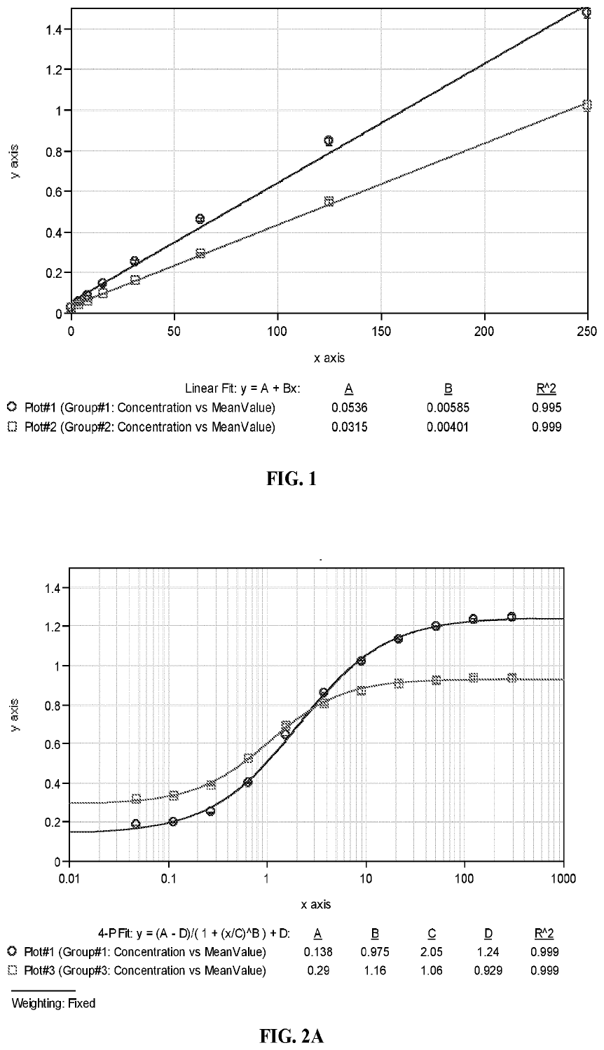

Under the same conditions, Hyphen BFXa has −31% lower chromogenic activity than HFXa based on μg/mL concentrations (FIG. 1). This observation indicated that the direct potency assay needs to be optimized with BFXa, because by matching mass or concentration with HFXa, BFXa would have less activity; whereas by matching activity, BFXa would have molar excess than HFXa and thus affect the FXa/Inhibitor ratio and the degree of inhibition in the assay. In addition, betrixaban was found to be less potent for the inhibition of BFXa. Therefore, the assay conditions would need further optimization on the concentrations of both BFXa and betrixaban (inhibitor).

### Comparison Between HFXa and Hyphen BFXa in the Direct Potency Assay

The initial setup for the direct potency units assay used similar concentrations of BFXa and HFXa as in the direct potency assay (FIG. 2). For HFXa mixture, the 2× stock solutions were made at 1.0 μg/mL for HFXa and 40 ng/mL betrixaban. The HFXa (1×)/betrixaban (1×) control mixture was made by mixing equal volumes (1:1), resulting in 0.5 μg/mL HFXa and 20 ng/mL betrixaban. 50 μL of the FXa/betrixaban mixture was added to the reaction in the direct potency assay.

For BFXa mixture, the stock solutions were made at 0.9 μg/mL (2.0 Units/mL) for BFXa and 200-400 ng/mL for betrixaban, respectively. The higher betrixaban stock concentrations were prepared in order to accommodate the volume requirements for preparing the BFXa/betriaban mixture. The BFXa (1×)/betrixaban (1×) mixture was made by mixing proper volumes of BFXa and betrixaban, resulting in 0.5 μg/mL BFXa and 20 ng/mL betrixaban in the mixture. 50 μL of the fXa/betrixaban mixture was added to the reaction in the direct potency assay.

As shown in FIG. 2(a), BFXa had lower absorbance compared to HFXa at the highest andexanet concentration, consistent with the observed lower chromogenic activity of BFXa. However, in the absence of andexanet, BFXa had higher background in the presence of betrixaban, indicating less inhibition of BFXa activity.

Based on these observations, the betrixaban concentration in the mixture was increased (1.5×, 2.0×) in order to reduce the background activity of BFXa in the absence of andexanet, while keeping the same concentration for BFXa (1×). As shown in FIG. 2(b-c). A 2-fold increase of betrixaban concentration reduced the BFXa background to the level similar to HFXa in the absence of andexanet. However, the maximum absorbance with BFXa (1×) was lower in the presence of the highest andexanet concentration than the control with HFXa (1×)/betrixaban (1×).

### Optimization of the BFXa Concentration in the Direct Potency Assay

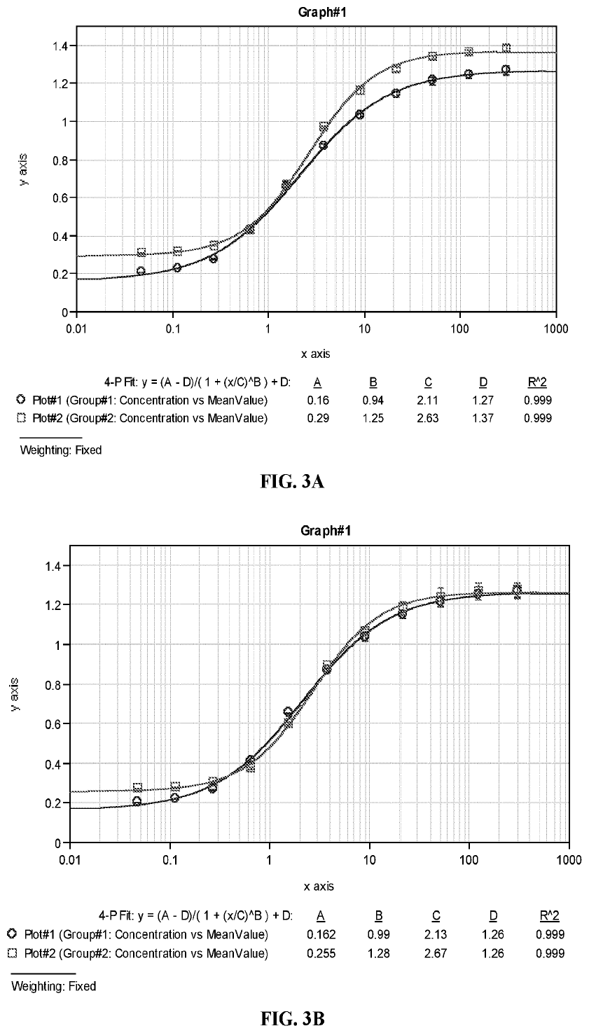

FIG. 3 shows the effect of varying the BFXa concentration on the maximum absorbance, while keeping the same concentration of betrixaban (2×). The mixture with BFXa (1.64×)/betrixaban (2×) showed an acceptable maximum absorbance but slightly higher than the HFXa control (FIG. 3a). Further adjustment of BFXa concentration with BFXa (1.45×)/betrixaban (2×) resulted in the final optimized conditions with comparable profiles between BFXa and the HFXa control (FIG. 3b).

The optimized conditions shown in FIG. 3b satisfy the intended assay setup to replace HFXa with BFXa in the original direct potency assay, with comparable profiles between BFXa and the HFXa control in terms of maximum and minimum absorbance, EC₅₀, and assay ranges. Similar assay procedures and criteria used in the direct potency assay can be used in the direct potency unit assay.

A change for the preparation of the BFXa/betrixaban mixture is therefore required to accommodate the changes made with the BFXa and betrixaban concentrations in the mixture. A sample protocol is shown as follows:
1) Preparation of BFXa (2.0 U/mL)
   Reconstitution of BFXa (Hyphen BE101K, 50 μg/vial) by adding 1.0 mL of purified water to the vial of Hyphen BFXa. This will result in 50 μg/mL of BFXa (111 Units/mL). Mix the content of the vial and keep the vial on ice.
   Dilute the reconstituted BFXa to 0.92 μg/mL (2.0 Units/mL) in Assay Buffer.
2) Preparation of betrixaban stock solution (200 μg/mL)
   Dilute the betrixaban stock to 200 μg/mL in Assay Buffer.
3) Preparation of BFXa/betrixaban mixture
   Mix 8 volumes of BFXa with 2 volumes of betrixaban, e.g. by combining 8.0 mL BFXa (0.92 μg/mL) and 2.0 mL betrixaban (200 μg/mL).
   Add the BFXa/betrixaban mixture (50 μL) to the Direct Potency units assay mixture.

The BFXa concentration/mass in the 50 μL mixture is 1.6 Units/mL and 0.08 Units, respectively. The betrixaban concentration/mass in the 50 μL mixture is 40 μg/mL and 2 μg, respectively.

Since the Hyphen BFXa used in the assay has been calibrated against the NIBSC BFXa (75/595), the Direct Potency units for andexanet alfa reference standard are traceable to an independent international reference standard. The Hyphen BFXa, further, can be used to calibrate HFXa reference samples.

A HFXa international reference standard [NIBSC code: 15/102], which became available recently, can be used similarly as the BFXa reference standard for HFXa calibration or used directly in the potency unit assay traceable to an independent international reference standard.

The contents of the articles, patents, and patent applications, and all other documents and electronically available information mentioned or cited herein, are hereby incorporated by reference in their entirety to the same extent as if each individual publication was specifically and individually indicated to be incorporated by reference.

Applicants reserve the right to physically incorporate into this application any and all materials and information from any such articles, patents, patent applications, or other physical and electronic documents.

The disclosure has been described broadly and generically herein. Each of the narrower species and subgeneric groupings falling within the generic disclosure also form part of the disclosure. This includes the generic description of the disclosure with a proviso or negative limitation removing any subject matter from the genus, regardless of whether or not the excised material is specifically recited herein. Other embodiments are within the following claims. In addition, where features or aspects of the disclosure are described in terms of Markush groups, those skilled in the art will recognize that the disclosure is also thereby described in terms of any individual member or subgroup of members of the Markush group.

1. A method for determining the activity of an andexanet sample, comprising:
admixing a test sample comprising andexanet with a mixture comprising a human factor Xa (HFXa) and a direct factor Xa (fXa) inhibitor, wherein the molar ratio of the fXa to the fXa inhibitor is from 0.2:1 to 0.3:1;
adding a chromogenic fXa substrate to the mixture, wherein the chromogenic fXa substrate is able to release a chromophore upon reaction with the HFXa;
detecting the amount of released chromophore; and
calculating, from the amount of the released chromophore, the activity of the andexanet sample in releasing the HFXa from the direct fXa inhibitor.

2. The method of claim 1, wherein the molar ratio of the HFXa to the fXa inhibitor is from 0.22 to 0.28.

3. The method of claim 1, wherein the molar ratio of the HFXa to the fXa inhibitor is from 0.24 to 0.26.

4. The method of any preceding claim, wherein the HFXa, after the test sample is added, has a concentration of about 8 to 14 nM.

5. The method of claim 4, wherein the HFXa, after the test sample is added, has a concentration of about 10 to 12 nM.

6. The method of any preceding claim, wherein the direct fXa inhibitor is selected from the group consisting of betrixaban, apixaban, rivaroxaban, edoxaban, otamixaban, letaxaban and eribaxaban.

7. The method of claim 6, wherein the direct fXa inhibitor is betrixaban.

8. The method of any preceding claim, wherein the chromogenic fXa substrate is spectrozyme-Xa.

9. The method of any preceding claim, further comprising validating the activity of the andexanet sample with a standard curve generated with a reference andexanet sample.

10. A method for determining the activity of an andexanet sample, comprising:
admixing a test sample comprising andexanet with a mixture comprising a bovine factor Xa (BFXa) and a direct factor Xa (fXa) inhibitor, wherein the molar ratio of the BFXa to the fXa inhibitor is from 0.15:1 to 0.25:1;
adding a chromogenic fXa substrate to the mixture, wherein the chromogenic fXa substrate is able to release a chromophore upon reaction with the BFXa;
detecting the amount of released chromophore; and
calculating, from the amount of the released chromophore, the activity of the andexanet sample in releasing the BFXa from the direct fXa inhibitor.

11. The method of claim 10, wherein the molar ratio of the BFXa to the fXa inhibitor is from 0.16 to 0.20.

12. The method of claim 10, wherein the molar ratio of the BFXa to the fXa inhibitor is from 0.17 to 0.19.

13. The method of any one of claims 10-12, wherein the BFXa, after the test sample is added, has a concentration of about 10 to 20 nM.

14. The method of claim 13, wherein the BFXa, after the test sample is added, has a concentration of about 14 to 18 nM.

15. The method of any one of claims 10-14, wherein the direct fXa inhibitor is selected from the group consisting of betrixaban, apixaban, rivaroxaban, edoxaban, otamixaban, letaxaban and eribaxaban.

16. The method of claim 15, wherein the direct fXa inhibitor is betrixaban.

17. The method of any one of claims 10-16, wherein the chromogenic fXa substrate is Spectrozyme-Xa.


SEQUENCE LISTING (4 sequences)

SEQ ID NO: 1 - protein, 393 aa, Homo sapiens
ANSFLEEMKKGHLERECMEETCSYEEAREVFEDSDKTNEFWNKYKDGDQCETSPCQNQGKCKDGLGEYTCTCLEGFEGKNCELFTRKLCSLDNGDCDQFCHEEQNSVVCSCARGYTLADNGKACIPTGPYPCGKQTLERIVGGQECKDGECPWQALLINEENEGFCGGTILSEFYILTAAHCLYQAKRFKVRVGDRNTEQEEGGEAVHEVEVVIKHNRFTKETYDFDIAVLRLKTPITFRMNVAPACLPERDWAESTLMTQKTGIVSGFGRTHEKGRQSTRLKMLEVPYVDRNSCKLSSSFIITQNMFCAGYDTKQEDACQGDAGGPHVTRFKDTYFVTGIVSWGEGCARKGKYGIYTKVTAFLKWIDRSMKTRGLPKAKSHAPEVITSSPLK

SEQ ID NO: 2 - protein, 359 aa, Artificial Sequence
  Description of Artificial Sequence Synthetic polypeptide
ANSFLFWNKYKDGDQCETSPCQNQGKCKDGLGEYTCTCLEGFEGKNCELFTRKLCSLDNGDCDQFCHEEQNSVVCSCARGYTLADNGKACIPTGPYPCGKQTLERIVGGQECKDGECPWQALLINEENEGFCGGTILSEFYILTAAHCLYQAKRFKVRVGDRNTEQEEGGEAVHEVEVVIKHNRFTKETYDFDIAVLRLKTPITFRMNVAPACLPERDWAESTLMTQKTGIVSGFGRTHEKGRQSTRLKMLEVPYVDRNSCKLSSSFIITQNMFCAGYDTKQEDACQGDAGGPHVTRFKDTYFVTGIVSWGEGCARKGKYGIYTKVTAFLKWIDRSMKTRGLPKAKSHAPEVITSSPLK

SEQ ID NO: 3 - protein, 105 aa, Artificial Sequence
  Description of Artificial Sequence Synthetic polypeptide
ANSFLFWNKYKDGDQCETSPCQNQGKCKDGLGEYTCTCLEGFEGKNCELFTRKLCSLDNGDCDQFCHEEQNSVVCSCARGYTLADNGKACIPTGPYPCGKQTLER

SEQ ID NO: 4 - protein, 254 aa, Artificial Sequence
  Description of Artificial Sequence Synthetic polypeptide
IVGGQECKDGECPWQALLINEENEGFCGGTILSEFYILTAAHCLYQAKRFKVRVGDRNTEQEEGGEAVHEVEVVIKHNRFTKETYDFDIAVLRLKTPITFRMNVAPACLPERDWAESTLMTQKTGIVSGFGRTHEKGRQSTRLKMLEVPYVDRNSCKLSSSFIITQNMFCAGYDTKQEDACQGDAGGPHVTRFKDTYFVTGIVSWGEGCARKGKYGIYTKVTAFLKWIDRSMKTRGLPKAKSHAPEVITSSPLK